(19) United States
(12) Patent Application Publication
(10) Pub. No.: US 2023/0200908 A1
(43) Pub. Date: Jun. 29, 2023

(54) COMPUTING PLATFORM FOR IMPROVED AESTHETIC OUTCOMES AND PATIENT SAFETY IN MEDICAL AND SURGICAL COSMETIC PROCEDURES

(71) Applicant: STANDARD OF CARE CORPORATION, Deerfield, IL (US)

(72) Inventors: Iliana E. SWEIS, Chicago, IL (US); Bryan C. CRESSEY, Chicago, IL (US)

(21) Appl. No.: 18/111,428

(22) Filed: Feb. 17, 2023

Related U.S. Application Data
(63) Continuation-in-part of application No. 17/447,914, filed on Sep. 16, 2021.
Application No. 17/447,914 is a continuation of application No. 16/399,916, filed on Apr. 30, 2019, now Pat. No. 11,123,140.
(60) Provisional application No. 62/664,903, filed on Apr. 30, 2018.

(51) Int. Cl. A61B 34/10; G16H 50/50; G16H 30/40

(52) U.S. Cl. CPC A61B 34/10; G16H 50/50; G16H 30/40; A61B 2034/101; G16H 20/40

(57) ABSTRACT

An electronic computer system classifies an anatomical target by obtaining a series of input images of the target; detecting a difference in a characteristic of the anatomical target across the series of input images; comparing, using a pattern recognition process, the difference in the characteristic across the series of input images to respective differences in characteristics across respective series of reference images; and classifying the anatomical target based on similarities with reference images analyzed during the comparing.

### RELATED APPLICATIONS

This application is a continuation-in-part of U.S. patent application Ser. No. 17/447,914, filed Sep. 16, 2021, which is a continuation of U.S. patent application Ser. No. 16/399,916, filed Apr. 30, 2019 and issued as U.S. Pat. No. 11,123,140 on Sep. 21, 2021, which claims priority to U.S. Provisional Patent Application No. 62/664,903, filed Apr. 30, 2018, each of which is hereby incorporated by reference in its entirety.

This application is related to U.S. patent application Ser. No. 15/162,952, filed May 24, 2016, entitled “Marking Template for Medical Injections, Surgical Procedures, or Medical Diagnostics and Methods of Using Same,” which is hereby incorporated by reference in its entirety.

### TECHNICAL FIELD

This application relates generally to computer technology and medical and/or surgical cosmetic procedures, including but not limited to methods and systems for using machine learning to improve aesthetic outcomes and patient safety.

### BACKGROUND

There is a continuing increase in the number of medical and surgical cosmetic procedures being performed, the most significant of which is facial injectables. These include two main categories: neuromodulators and soft tissue fillers. The potential complications of soft tissue fillers are serious, and may be permanent. For instance, cases of stroke and blindness have been reported with the use of soft tissue fillers. The reported cases do not include cases that are performed under less than ideal conditions and go unreported.

There are core physicians who perform these injections, including plastic surgeons, dermatologists, facial plastic surgeons, and oculoplastic surgeons. These practitioners are considered to be properly trained injectors. Many physicians delegate the injections to nurses in their practice (nurse injectors) who have attended courses and learned to inject. In addition to the core physicians, many non-core physicians (internists, family practice, gynecologists, anesthesiologists, etc.) have opened medical spas, and a significant portion of their business is injectables. More importantly, many injectors are not physicians or even nurses.

Due to the wide range of practitioner backgrounds in the field of cosmetic procedures, as well as the potential for serious complications, there is a need for improved aesthetic outcomes and increased safety of patients who are seeking such treatments.

### SUMMARY

Implementations described in this specification are directed to providing a computing platform for use by medical providers who treat patients seeking cosmetic procedures. In some implementations, the platform stores and analyzes a plurality of images of faces (e.g., several thousand faces), and/or information associated with images of faces, and uses machine learning and/or pattern recognition (collectively, “machine learning”) to create treatment plans and recommendations in order to (i) reduce errors for practitioners and (ii) achieve better outcomes for patients.

In one aspect of the application, a method of creating safe and accurate treatment plans is implemented at a computer system having one or more processors and memory storing one or more programs for execution by the one or more processors. The method includes obtaining an input image of a face; comparing, using a machine learning process, one or more aspects of the input image to corresponding aspects of a plurality of reference images; obtaining, based on a result of the comparing, supplemental information associated with one or more additional characteristics of the face; and creating a treatment plan based on the input image and the supplemental information.

In accordance with some aspects of this application, a computer system includes memory storing instructions for causing the computer system to perform any of the methods described herein.

Further, in accordance with some aspects of this application, instructions stored in memory of a computer system include instructions for causing the computer system to perform any of the methods described herein.

Other embodiments and advantages may be apparent to those skilled in the art in light of the descriptions and drawings in this specification.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is a system diagram of a computing platform and its context, in accordance with some embodiments.

FIGS. 2-7 are diagrams illustrating data structures used by the machine learning module of FIG. 1, in accordance with some embodiments.

FIG. 8 is a flow diagram illustrating a method for providing customized treatment plans in accordance with some embodiments.

FIGS. 9-11 are diagrams of various patient images with markers provided for guidance, in accordance with some embodiments.

FIGS. 12A-14B are diagrams of various spatial measurements of facial landmarks and corresponding mathematical standards in accordance with some implementations.

FIGS. 15A-15B are flow diagrams illustrating operations for providing customized treatment plans based on spatial measurements and corresponding mathematical standards in accordance with some implementations.

FIGS. 16-17 are diagrams illustrating classification and growth data structures used by the machine learning module of FIG. 1, in accordance with some embodiments.

FIG. 18 is a flow diagram illustrating a method for classifying an anatomical target in accordance with some embodiments.

Like reference numerals refer to corresponding parts throughout the drawings.

### DESCRIPTION OF IMPLEMENTATIONS

Implementations described in this specification are directed to providing a computing platform for use by medical providers who treat patients seeking cosmetic procedures. In some implementations, the platform stores and analyzes a plurality of images of faces (e.g., several thousand faces), or information associated with images of faces, and uses machine learning to create treatment plans and recommendations in order to (i) reduce errors for practitioners and (ii) achieve better outcomes for patients.

The potential complications of neuromodulators, such as a droopy eyelid and facial asymmetry, are self-limiting and reversible. The neuromodulator effect usually diminishes by two months, and is usually gone by three months.

However, the potential complications of fillers are more serious, and may be permanent. This is due to the fact that these products are not water soluble. The facial blood supply is quite extensive, and vessels communicate with one another through an arcade. It is possible for the needle to be accidentally placed through a blood vessel during injection, which could result in compromising the blood flow to the area supplied by that vessel. This may lead to a temporary change in color or in tissue death in the treated area leading to a scab and/or permanent scar formation. In some cases, the product can be carried in a vessel that reaches the brain or the eye, which may lead to a stroke or blindness.

The implementations described herein improve aesthetic outcomes and increase the safety of patients who are seeking such treatments. In some implementations, the computing platform achieves these outcomes by using machine learning, in combination with beauty and safety databases, facial topographical analysis, and multispecialty medical expertise to create treatment plans for patients seeking aesthetic improvements (e.g., to the face).

In some implementations, the computing platform utilizes visual sensors to gather facial data in order to develop facial recognition and further utilizes machine learning to understand concepts of facial youthfulness and facial beauty. The platform combines that data with topographical facial analysis and the expertise of a large group of plastic surgeons, dermatologists, and other cosmetic specialists to create and recommend safe treatment protocols and algorithms for enhancing the facial features according to documented, artistic and machine-learned concepts of youth and facial beauty.

In some implementations, the computing platform utilizes facial recognition and machine learning to determine whether or not the patient is a good candidate for injectables or whether surgery is a more appropriate option. In some implementations, the computing platform determines whether a patient is a proper candidate for elective procedures based on their answers to a preliminary evaluation (e.g., a questionnaire with a built-in scale assessing psychological stability and possible Body Dysmorphic Syndrome).

In some implementations, the knowledge base for the computing platform is initially provided by one or more of: plastic surgeons, facial plastic surgeons, oculoplastic surgeons, dermatologists, laser specialists, psychiatrists, anatomists, and/or research and development experts in the fields of neuromodulators and facial fillers.

In some implementations, the computing platform has at least two major subject areas for machine learning: (1) enhancing facial features (e.g., through injectables and/or surgery), and (2) reversing signs of aging (e.g., through injectables and/or surgery).

Embodiments of the computing platform disclosed herein increase the safety of injections being performed on the patient, improve the aesthetic quality and outcome of injections being performed on the patient, do not require a core facial aesthetic physician to implement, allow use by a nurse or doctor who is not a core facial aesthetic specialist, continue learning and adapt as new concepts of facial beauty evolve over time, and/or continue learning and adapt as new injectable products, new lasers, new skin care lines and/or new surgical procedures are developed.

Embodiments of the computing platform disclosed herein provide specific protocols using neuromodulators and soft tissue fillers with detailed guidance as to how to inject these in specific locations (e.g., facial locations) to obtain excellent aesthetic outcomes while promoting a high degree of patient safety by accounting for nerves, blood vessels and other vital structures. In some implementations, the computing platform provides recommendations for further skin enhancement using laser treatments and medical grade skin care.

In some implementations, a practitioner (e.g., nurse or doctor) uploads, or otherwise inputs, one or more photos of a patient's face. In some implementations, the computing platform first validates the image(s), for example, by indicating whether the image(s) meet a threshold level of quality and/or satisfy particular angles.

In some implementations, the computing platform analyzes the images to determine skin type (e.g., Fitzpatrick Classification (Type I through VI)) and/or specific details of the face and neck relative to documented and learned concepts of youth and facial beauty as defined within a particular race, ethnicity, gender, and/or age. For example, the computing platform analyzes one or more of:
Facial Structure (e.g., adequate or deficient bone structure based on external bony landmarks, adequate or deficient soft tissue volume in upper third, mid-third and lower third of face);
Rhytids due to muscle movement (e.g., with classification between dynamic rhytids and static rhytids);
Rhytids due to loss of skin constituents (e.g., collagen, elastin and hyaluronic acid)
Solar elastosis (e.g., percentage of face and neck affected by sun damage, solar lentigines, redness, prominent vessels), and/or
Extent of descent of the superficial musculoaponeurotic system (SMAS), platysma, and overlying tissues.

In some implementations, the computing platform then analyzes the images to determine how to improve the face based on documented and learned concepts of facial beauty. For example, the computing platform utilizes 3D facial imaging, Smart Grid imaging (e.g., as disclosed in U.S. patent application Ser. No. 15/162,951, which is incorporated by reference in its entirety), and facial vessel visualization technology to outline the accurate and safe placement of soft tissue fillers in the face.

In some implementations, the computing platform instructs the injector step-by-step using neuromodulators and soft tissue filler injection techniques that implement a high degree of patient safety. For example, the computing platform identifies for the injector one or more of:
Degree of difficulty of injection and level of injector experience required;
Degree of patient satisfaction with particular injection procedures (e.g., high patient satisfaction; relatively predictable outcome; variable patient satisfaction; sometimes unpredictable outcome); and/or
Risk of complications (e.g., low, medium, high)

In some implementations, the computing platform analyzes the image(s) to determine one or more of:
What injection products to use;
Where to place each of the injections (e.g., using Smart Grid imaging and/or vessel visualization technology);
What the ideal sequence of injections is;
How much product to use in each facial region or injection site;
What depth of injection is safest in different facial regions;
What technique of injection is necessary for each region; and/or
Visualization of partial correction and complete correction of the face prior to performing the injections.

In some implementations, the computing platform performs one or more of the above determinations by:
Identifying proper candidates for injectables;
Identifying the most likely anatomy of the vessels, nerves, fat, facial muscles, bony structure, and parotid glands in the face;
Recommending the proper neuromodulator and soft tissue filler for each area to be treated;
Recommending the correct sequence of the injections;
Recommending the proper volume of the injections;
Advising how to avoid pitfalls/complications;
Providing technique videos;
Demonstrating to the patient what multilevel injections can achieve at each level of injection (e.g., after a first injection, second injection, and so forth); and/or
Addressing the risks associated with each injection.

In some implementations, the computing platform performs a surgical evaluation of the face to determine a proper course of action with non-surgical procedures. For example, injectables may be used to get as close as possible to a surgical result. In some implementations, the computing platform evaluates one or more Aesthetic Facial Units (e.g., forehead, eyelids, nose, cheeks, lips, chin, and pinna) in terms of what is deficient and what is in excess, what is missing and what is an undesirable trait (e.g., low lying eyebrows, deficient cheek bones, deficient chin projection, excess maxillary show, presence of jowls, etc.), and determines the proper treatment plan.

For example, instead of looking at wrinkles in the forehead to determine where and how much of an injectable to use to treat the wrinkles, the computing platform evaluates the forehead as a unit and examines the brow position, loss of volume, extent and location of wrinkles, and asymmetry. The computing platform then creates a treatment plan that includes recommending the proper dose and placement of one or more particular injectables, as well as the proper sequence of injection, to create a more aesthetic brow position and to soften the forehead wrinkles and restore volume (similar to what could alternatively be achieved through a surgical brow lift).

Applying a surgical approach to non-surgical techniques is unique in that it increases the safety and aesthetic results of current techniques. Stated another way, the computing platform dictates non-surgical treatment recommendations in a medical/surgical discipline.

In some implementations, the computing platform performs a surgical evaluation of various parts of the body to determine a proper course of action using surgical procedures. Example surgical procedures include surgery of the breast, nose shaping, and flap reconstruction. For each type of surgical procedure, the computing platform evaluates one or more physical characteristics (e.g., presented in images and/or alternative media), and creates and recommends one or more treatment plans as described below. To be clear, example processes described in this specification for creating and recommending treatment plans apply equally to surgical procedures as well as to non-surgical procedures.

While implementations described herein may refer to the face or regions surrounding the face (e.g., nose, neck), these references are exemplary in nature, and those skilled in the art will appreciate from the present disclosure that various other parts of the human body have not been illustrated for the sake of brevity, and so as not to obscure more pertinent aspects of the example implementations disclosed herein. As such, examples described herein referring to the face should be construed as also being applicable to any other part of the body.

### Computing Platform Architecture

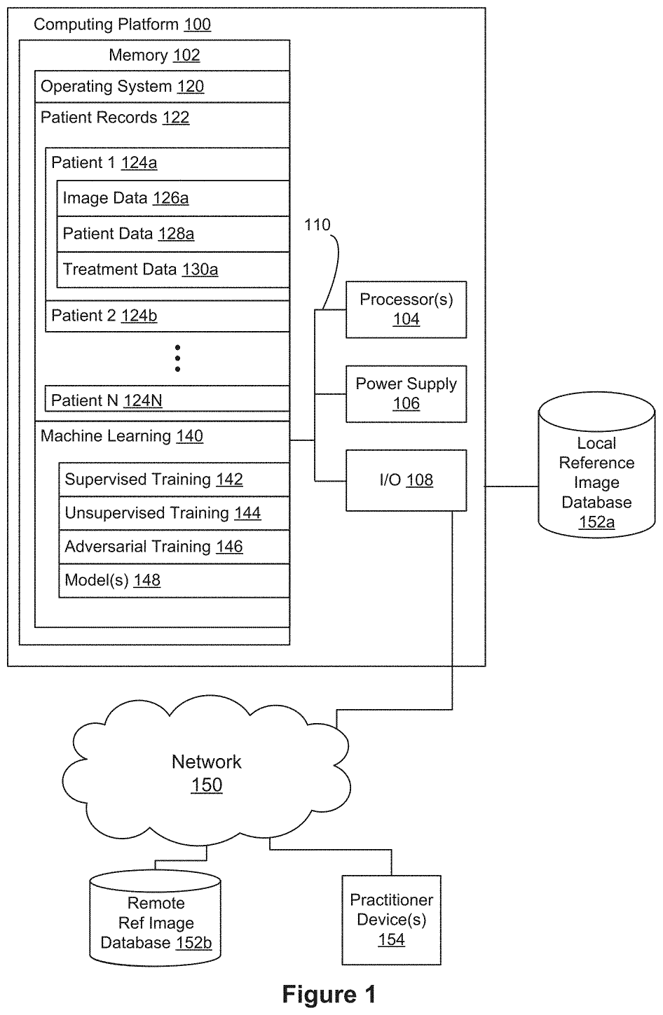

FIG. 1 is a system diagram of a computing platform 100 (also referred to herein as a “machine learning system”), in accordance with some embodiments. The computing platform 100 typically includes a memory 102, one or more processor(s) 104, a power supply 106, an input/output (I/O) subsystem 108, and a communication bus 110 for interconnecting these components.

The processor(s) 104 execute modules, programs, and/or instructions stored in the memory 102 and thereby perform processing operations.

In some embodiments, the memory 102 stores one or more programs (e.g., sets of instructions) and/or data structures, collectively referred to as “modules” herein. In some embodiments, the memory 102, or the non-transitory computer readable storage medium of the memory 102 stores the following programs, modules, and data structures, or a subset or superset thereof:
an operating system 120;
patient records 122, including data for individual patients 124, which includes image data 126 (e.g., one or more images of the patient's face), patient data 128 (e.g., evaluation data such as questionnaire answers, age, gender, expectations, desired outcomes, and so forth), and/or treatment data 130 (e.g., a recommended procedure to be performed in accordance with the patient's desired outcome and image data, as determined by the computing platform); and
a machine learning module 140 that uses supervised training module 142, unsupervised training module 144, and/or adversarial training module 146 to generate one or more facial models 148 (e.g., by analyzing reference images corresponding to a plurality of faces and procedures).

The above identified modules (e.g., data structures and/or programs including sets of instructions) need not be implemented as separate software programs, procedures, or modules, and thus various subsets of these modules may be combined or otherwise re-arranged in various embodiments. In some embodiments, the memory 102 stores a subset of the modules identified above. In some embodiments, a local reference image database 152a and/or a remote reference image database 152b store a portion or all of one or more modules identified above. Furthermore, the memory 102 may store additional modules not described above. In some embodiments, the modules stored in the memory 102, or a non-transitory computer readable storage medium of the memory 102, provide instructions for implementing respective operations in the methods described below. In some embodiments, some or all of these modules may be implemented with specialized hardware circuits that subsume part or all of the module functionality. One or more of the above identified elements may be executed by one or more of the processor(s) 104. In some embodiments, one or more of the modules described with regard to the memory 102 is implemented in the memory of a practitioner device 154 and executed by processor(s) of the practitioner device 154.

In some embodiments, generating a facial model 148 includes generating a regression algorithm for prediction of continuous variables (e.g., perspective transformation of a reference image and/or a more complex transformation describing morphing of facial images).

In some embodiments, the I/O subsystem 108 communicatively couples the computing platform 100 to one or more devices, such as a local reference image database 152a, a remote reference image database 152b, and/or practitioner device(s) 154 via a communications network 150 and/or via a wired and/or wireless connection. In some embodiments, the communications network 150 is the Internet.

The communication bus 110 optionally includes circuitry (sometimes called a chipset) that interconnects and controls communications between system components.

Typically, a system for recommending treatment procedures includes a computing platform 100 that is communicatively connected to one or more practitioner devices 154 (e.g., via a network 150 and/or an I/O subsystem 108). In some embodiments, the system receives patient records 122 (e.g., from a practitioner device 154 that captures or otherwise receives an image of a patient 124). For example, the patient data includes an image 126 and additional data 128 corresponding to the patient (e.g., desired outcome data). Practitioner device 154 is, for example, a computing system or platform (e.g., a laptop, computer, physical access system, or a mobile device) of a doctor or nurse.

### Training the Computing Platform

In some implementations, an image database 152 of the computing platform stores a plurality of images of faces (e.g., hundreds, thousands, or more), or information associated with images of faces. In some implementations, each image (or information associated with each image) in the database is associated with a treatment plan, including one or more of (1) specific agents and amounts/units that were used or would be used, (2) locations for each injection, and (3) a proper sequence of injection, as described above. In some implementations, the treatment plans that are associated with each facial image correspond with actual treatment plans that were performed on the subject of the image. Alternatively, the treatment plans that are associated with each facial image correspond with suggested treatment plans, wherein the suggestions are based on various physical aspects of the face, such as shapes of facial features, positions of facial features with respect to other features, and/or locations of anatomical obstructions (e.g., nerves and blood vessels).

In some implementations, machine learning is used to identify commonalities in certain types of facial features in the context of their associated treatment plans. In other words, by using machine learning, the computing platform recognizes relationships between facial features and particular aspects of treatment plans. In some implementations, machine learning is applied to these relationships to extend the computing platform's basis for determining a treatment plan (also referred to herein as creating, generating, forming, or building a treatment plan) and making recommendations in accordance with the determined treatment plan. For example, the computing platform's basis for determining a treatment plan is extended to images of faces that have not been analyzed at the time of the treatment plan determination (referred to herein as new faces). As such, upon analyzing a new face, the computing platform identifies the most likely set of steps or processes for treatment of the new face based on the previously identified relationships between facial features in common with particular faces stored in the database and treatment plans corresponding to those particular faces.

In some implementations, in order to respect patient privacy, the computing platform deletes raw patient images after the platform has developed algorithms or models for creating treatment plans. Alternatively, in order to respect patient privacy, the facial images that are used for training are not obtained from patients, and instead are obtained from other sources (e.g., an online face repository).

In some implementations, facial images in the database are associated with “after” versions (which are also stored in the database) showing what the face looks like, or would look like, upon completion of treatment. In some implementations, the “after” image of a patient's face is obtained upon completion of an actual treatment. Alternatively, facial images obtained from non-patient sources are edited to show an “after” version of what the face would look like after a particular treatment procedure. Regardless of the source, the “after” images are stored in the database and are associated with the “before” images in the database, and the computing platform uses machine learning to determine what a new face would look like upon completion of a particular treatment procedure. In some implementations, the determined “after” image for a new face is displayed to the patient for the patient's consideration in electing whether to proceed with the particular treatment plan. In some implementations, the determined “after” image for a new face is displayed to the practitioner in order to assist the practitioner in carrying out the particular treatment plan, or in order to assist the practitioner in recommending alternative treatment plans.

In some implementations, the computing platform also considers, in addition to facial features, one or more additional characteristics associated with the face (or associated with the patient to which the face belongs; e.g., patient data 128), where the one or more additional characteristics are selected from the group consisting of: gender, age, concerns, goals, and physical conditions of various aspects of the face. In some implementations, these additional characteristics are also stored in the database and associated with each face, and the computing platform uses machine learning to recognize patterns and relationships between the faces and the additional characteristics.

In some implementations, the computing platform develops a plurality of base algorithms, directed to each additional characteristic, for creating treatment plans. Patients may present individual characteristics on a gradient. For example, for age: not too young, not too old, but somewhere in the middle; for goals: not too aggressive of a procedure, not too passive of a procedure, but somewhere in the middle; and so forth. Accordingly, in some implementations, the computing platform merges one or more of the base algorithms into a combined algorithm based on the gradients of the base algorithms. In other words, the combined algorithm creates a treatment plan based on a gradient of each base algorithm's treatment plan.

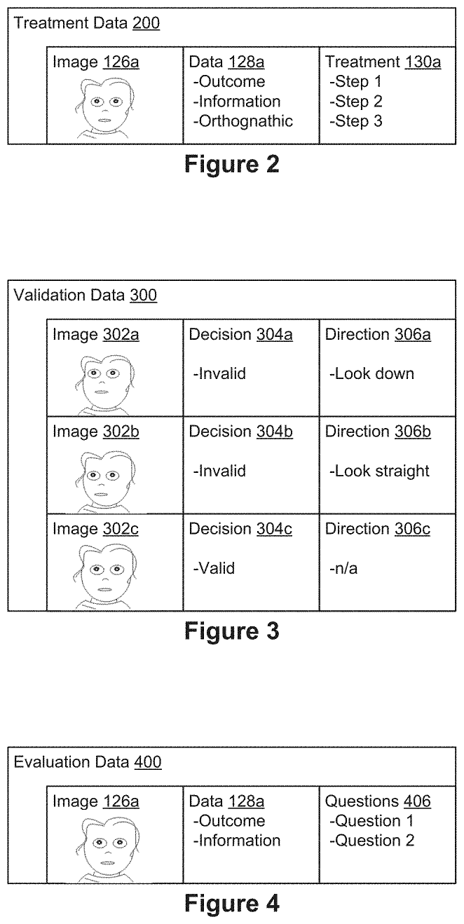

FIGS. 2-7 are diagrams showing data structures for machine learning processes in accordance with some embodiments. Embodiments of the machine learning module 140 train one or more facial models 148 in accordance with each figure, as explained in more detail below. In some embodiments, the image data and/or other data that make up the respective data structures is stored in a local or remote database 152. Alternatively, the image and/or other data that make up the respective data structures is stored in memory 102 of the computing platform 100. In any case, the computing platform inputs the data in a respective structure into a training module (e.g., 142, 144, or 146) for development of a respective model 148, as described below.

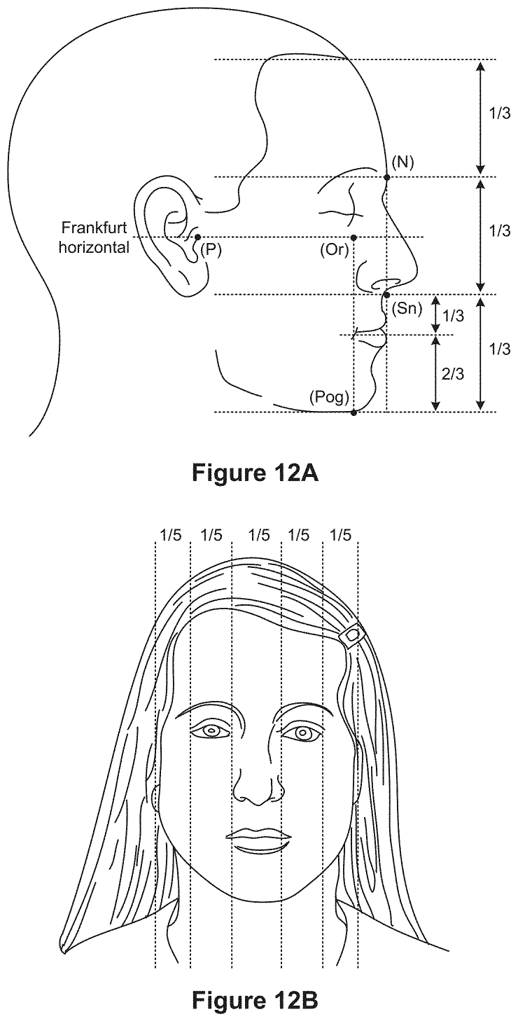

FIG. 2 is a diagram of treatment data 200 corresponding with patient 124a in FIG. 1 in accordance with some embodiments. For each patient, an image 126 (alternatively referred to herein as image data 126) is stored, along with non-image data 128 such as desired outcomes, patient expectations, physical characteristics of the patient (e.g., age, gender, and so forth), anatomical information, and/or spatial measurement data (e.g., as described below with reference to FIGS. 12-14). When training a treatment model 148 for determining a recommended procedure for the particular patient, treatment data 130 is also stored and associated with the image 126 and data 128. In some embodiments, image data 126 includes a plurality of images of the patient, including images taken from different angles, and/or images showing different areas of the face. For training purposes, the image data 126 and non-image data 128 serve as a machine learning input, and the corresponding treatment data 130 serves as an input label for that machine learning input. With a set of inputs (e.g., hundreds, thousands, or more), machine learning module 140 generates a treatment model 148 for determining a treatment plan 130 for new sets of images 126 and data 128.

FIG. 3 is a diagram of validation data 300 corresponding with patient 124a in FIG. 1 in accordance with some embodiments. For each patient, image data 302 is stored, along with a validation decision (e.g., valid or invalid). The decision is based on the angles and areas necessary to be included in an image for a given procedure (described in more detail below). For training purposes, each image constituting image data 302 serves as machine learning input, and the corresponding validation decision 304 serves as an input label for that machine learning input. With a set of inputs, machine learning module 140 generates a validation model 148 for determining whether a subsequently received image is valid. Optionally, directions 306 are also included as input labels for training purposes. Accordingly, the validation model 148 would determine (i) whether a subsequently received image is valid, and (ii) if invalid, the reason the image was invalid. For example, in FIG. 3, the validation model determines that image 302a is invalid (304a) because certain facial features are not depicted in the expected locations with respect to one another. The validation model determines that if the patient tilts his or her head downward (306a), upward, to the left, or to the right, a subsequent image would likely produce a more useful result.

FIG. 4 is a diagram of evaluation data 400 corresponding with patient 124a in FIG. 1 in accordance with some embodiments. For each patient, image data 126 and non-image data 128 are stored as described above. For training purposes, image data 126 and non-image data 128 for the patient serves as a machine learning input, and one or more corresponding evaluation questions 406 (described in more detail below) serve as input label(s) 406 for that machine learning input. With a set of inputs, machine learning module 140 generates an evaluation model 148 for determining a set of evaluation questions to propose to the patient based on his or her image data 126 and non-image data 128. In some embodiments, training also includes an additional step (not shown), wherein the image data 126, non-image data 128, and responses to the questions 406 serve as machine learning inputs, and one or more additional questions serve as input label(s) for that machine learning input. As such, the evaluation model would first output an initial set of questions based on the image data 126 and non-image data 128, and responses to the initial set of questions would serve as an input to a secondary evaluation model, which would output a subsequent set of questions based on the responses to the initial set of questions. In some embodiments, depending on the complexity of the evaluation, additional steps are implemented as described above, from which successive sets of questions are generated based on responses to the prior questions.

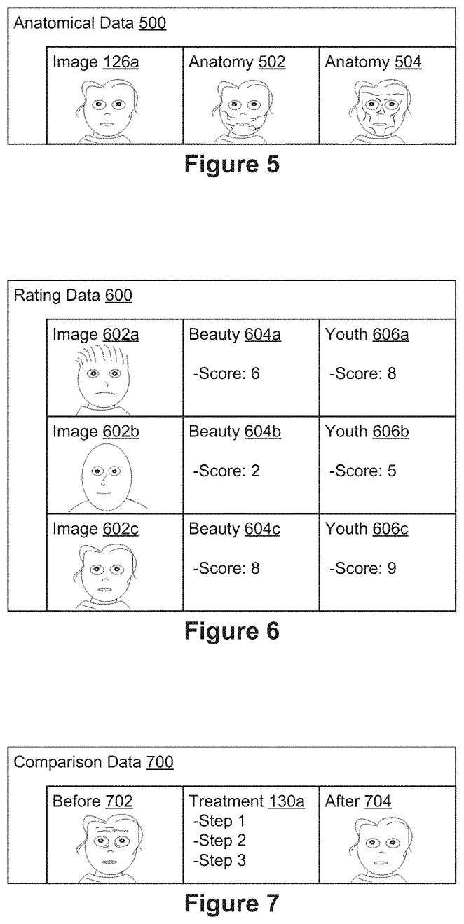

FIG. 5 is a diagram of anatomical data 500 corresponding with patient 124a in FIG. 1 in accordance with some embodiments. For each patient, image data 126 is stored as described above. For training purposes, image data 126 serves as a machine learning input, and anatomical data (e.g., one or more anatomy images 502 and/or 504) serves as input label(s) for that machine learning input. With a set of inputs, machine learning module 140 generates an anatomical model 148 for determining anatomical data (described in more detail below) associated with the patient based on the patient's image data 126. Optionally, non-image data 128 also serves as machine learning input.

FIG. 6 is a diagram of rating data 600 corresponding to several example input images in accordance with some embodiments. For each input image 602, a beauty score 604 and/or a youth score 606 (each described in more detail below) are assigned. For training purposes, each image 602 serves as a machine learning input, and the beauty 604 and/or youth 606 scores serve as input label(s) for that machine learning input. With a set of inputs, machine learning module 140 generates an anatomical model 148 for assigning beauty and/or youth scores associated with input images. In some embodiments, the scores are included in the non-image data 128 for a patient and included as inputs in the treatment model (FIG. 2).

FIG. 7 is a diagram of comparison data 700 corresponding with patient 124a in FIG. 1 in accordance with some embodiments. For each patient, image data 702 from before a particular procedure (e.g., treatment plan 130) is stored as described above. Additional image data 704 of the patient is stored after the procedure (e.g., immediately after the procedure, or after an appropriate recovery period). For training purposes, a “before” image 702 and corresponding treatment data 130 serve as a machine learning input, and an “after” image 704 serves as an input label for that machine learning input. With a set of inputs, machine learning module 140 generates a comparison model 148 for predicting what a patient will look like (704) after having undergone a particular procedure. Alternatively, a “before” image 702 and a requested outcome (e.g., a specific facial change, such as larger cheekbones, as stored in data 128) serve as a machine learning input, and an “after” image 704 serves as an input label for that machine learning input. That way, a patient may request a particular procedure, and the computing platform displays an image of the patient that projects what the patient will look like after having completed the procedure, based on the comparison model 148.

For each of the machine learning diagrams described above, machine learning module develops respective models 148 using supervised training (142), unsupervised training (144), and/or adversarial training (146). For supervised training, a practitioner manually assigns labels for respective inputs. For example, a practitioner:
assigns a particular treatment plan 130a for patient 126a according to the image data 126 and data 128 for that patient (FIG. 2);
assigns validation decisions 304 and corresponding directions 306 based on various input images 302 (FIG. 3);
determines appropriate questions and/or evaluation steps 406 to take based on image data 126 and/or non-image data 128 of a patient (FIG. 4);
predicts various anatomical obstructions and adds indicators of those obstructions to anatomical images 502/504 for a particular patient 126 (FIG. 5);
assigns beauty 604 and/or youth 606 scores for various images of patients and/or nonpatients 602, such people whose faces represent societal norms of beauty and/or youth (FIG. 6); and/or
chooses representative “after” images 704 for patients who have undergone particular procedures 130 (FIG. 7).

In some embodiments, supervised training module 142 facilitates manual labeling (as described above) by displaying successive input images to a practitioner (e.g., on a display on practitioner device 154), and receiving the manually entered input labels (e.g., from an input device via I/O module 108).

In some embodiments, after an initial learning process is complete, and models have been trained based on a plurality of inputs and corresponding labels, unsupervised training module 144 and/or adversarial training module 146 continue the training process by refining the models based on subsequently obtained images and data. In some embodiments, the computing platform obtains the subsequent images and data from an external source, such as an image gallery on the Internet. In some embodiments, training modules 144 and/or 146 periodically use subsequently obtained patient images to refine the models 148.

In some embodiments, machine learning module stores the input data and input labels as a pair (x, y), wherein x is the input data and y is the label. For some of the training embodiments described above, however, there are two or more inputs, or there are two or more labels. For these embodiments, the machine learning module trains the various models using a tuple (x1, x2, y) for embodiments with multiple input fields (e.g., image data and non-image data). The machine learning module trains the various models using a tuple (x, y1, y2) for embodiments with multiple labels (e.g., beauty score and youth score). Those skilled in the art will appreciate from the present disclosure that various other combinations of input (x) and label (y) data may be used by the machine learning module, depending on the training application. These other combinations have not been illustrated for the sake of brevity, and so as not to obscure more pertinent aspects of the example implementations disclosed herein.

### Treatment Plan Creation

In some implementations, an image of the patient's face or other portion of the user's body is uploaded to the computing platform, the computing platform creates one or more treatment plans 130, and presents one or more treatment recommendations based on the one or more treatment plans 130. In some implementations, the treatment plans include: (1) one or more specific agents and amounts/units to inject, (2) the locations for each injection, and (3) the sequence of injection (the order in which individual injections should take place). In some implementations, the computing platform also displays an “after” image of the patient's face (or other body portion), detailing what the patient's face (or other body portion) is predicted to look like after the procedure.

In some implementations, the computing platform obtains additional information associated with the patient (e.g., by asking questions, displaying prompts, and so forth). The substance of the questions and the order of the questions depend on the facial features and answers to previous questions. In some implementations, a first question seeks to determine the patient's concerns and/or goals, and a second question seeks to determine if the patient has a particular physical condition associated with an area that the patient wants to be treated. Treatment plans may be influenced by answers to these questions.

In one example, the patient's goal is to treat forehead lines. In accordance with the patient's goal, the computing platform asks a question relevant to possible treatment options. In this scenario, the computing platform may ask if the patient has frontalis hyperactivity. If the answer is yes, the computing platform determines that the patient's forehead lines cannot be treated because treatment would result in a dropping of the brow. On the other hand, if the answer is no, the computing platform determines that the patient's forehead lines can be treated. In addition, the computing platform creates and recommends a specific treatment plan as described above, and optionally, displays an “after” image for the patient to consider before electing to pursue the treatment plan.

In some implementations, subsequent prompts for additional treatment are displayed based on previous treatment areas. For example, the computing platform may determine that patients who elect to receive forehead line treatment usually also elect to receive eyelid treatment, and accordingly, the computing platform asks whether the patient would be interested in recommendations for eyelid treatment plans. In some implementations, one or more “after” images are constructed and displayed to the patient and/or the practitioner in order to assist in these treatment decisions.

In some implementations, from the patient's and/or the practitioner's point of view, the various implementations described herein demonstrate (1) the patient's current condition (e.g., what the patient looks like) at the time of consultation, (2) what the patient can look like after one or more customized treatment plans, and (3) the exact steps that would need to be taken in order to safely and accurately treat the patient.

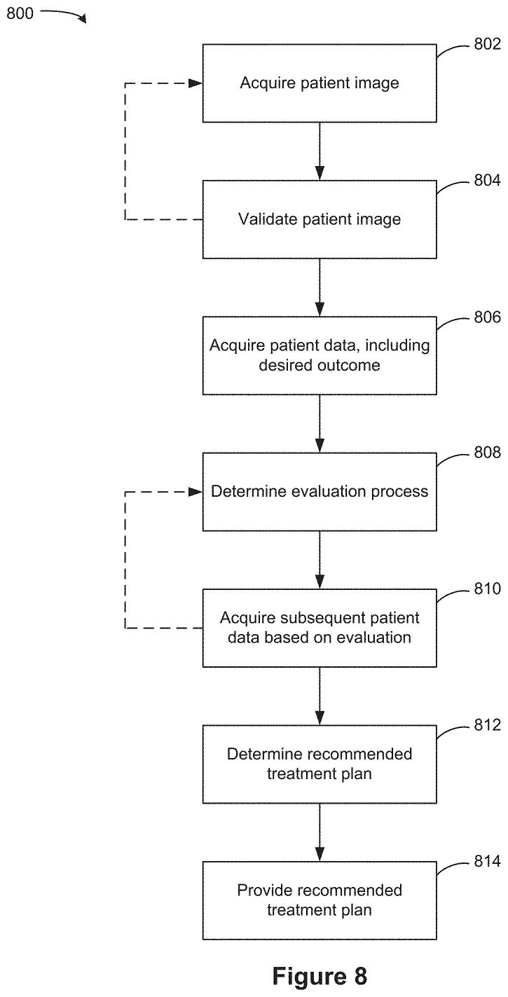

FIG. 8 is a flow diagram illustrating a method 800 for providing a customized treatment plan 130 to a patient, in accordance with some embodiments. The method is performed at a computing platform 100 (also referred to herein as a computer system), a local or remote image database 152, and/or a practitioner device 154. For example, instructions for performing the method are stored in the memory 102 and executed by the processor(s) 104 of the computer system 100. In some embodiments, part or all of the instructions for performing the method are stored in memory and executed by processor(s) of the practitioner device 154. In FIG. 8, dotted lines are used to indicate optional operations.

The system acquires (802) one or more images of the patient (e.g., image data 126). In some embodiments, a user interface of a display of the system 100 or device 154 displays a prompt for an image of the patient's face (or any body part undergoing cosmetic treatment). The practitioner captures the image using an imaging sensor (e.g., a camera) communicatively coupled, or capable of being communicatively coupled, to the system 100. The system receives the captured image and stores it in memory (e.g., image data 126a in memory 102). In some embodiments, the system 100 prompts the practitioner to obtain images of (i) the full face and neck in repose in three views (frontal, 45° angle, 90° angle); (ii) the full face and neck while smiling in three views (frontal, 45° angle, 90° angle); (iii) the full face and neck with the head tilted downward in three views (frontal, 45° angle, 90° angle); and/or (iv) a top-down view to assess malar region asymmetry. In some embodiments, the system 100 prompts the practitioner to obtain (i) frontal photos of the upper third of the face in repose; (ii) frontal photos of the upper third of the face with animation (e.g., frown, brow elevation, smile); (iii) oblique photos of the upper third of the face with maximum smile; (iv) photos of the lower third of the face in repose in three views (frontal, 45° angle, 90° angle); and/or (v) frontal photos of the lower third of the face with animation (frown, pursing of lips, smile).

In some embodiments, the system validates (804) the image before proceeding. Alternatively, the system validates the image after a subsequent step, or in some embodiments, does not perform a validation step. In some embodiments, the system validates the image using a validation model 148. Additionally or alternatively, the system validates the image by analyzing spatial features of particular areas of the face, such as distances, offsets, angles, and/or symmetries, and determining (e.g., based on the validation model 148) whether the system can rely on the image in further steps in accordance with the analysis. Additionally or alternatively, the system analyzes one or more of: image resolution, pan, tilt, zoom level, subject placement, and/or light levels to determine whether the system can accurately rely on the image in further steps. In some embodiments, the system simply uses the validation model 148 to determine a validation result.

In some embodiments, if an image does not pass the validation requirement, the system prompts the practitioner to obtain another image. Optionally, the prompt includes instructions (e.g., 306) as a result of applying the validation model 148.

In some embodiments, the system prompts the practitioner to obtain another image, regardless of the validation result. For instance, certain procedures (e.g., procedures requested by a patient or recommended by a treatment plan 130) require a plurality of views of a particular area of the face, captured from different angles. In some embodiments, the system obtains a plurality of images including different views, regardless of the procedure. Alternatively, the system only obtains images including views that are necessary for the particular procedure(s) that are requested or recommended. In some embodiments, the system includes instructions for the patient to move a particular part of the face in a certain way for one or more successive images (e.g., movements such as raising the eyebrows, smiling, flexing the neck, and so forth). For these embodiments, the system 100 stores successive patient images together as image data 126 in memory 102.

Upon receiving the requisite number and type of images, the system acquires (806) additional patient data (e.g., data 128). In some embodiments, the system acquires this data before acquiring the image(s), or concurrently to acquiring the image(s). The patient data includes physical characteristics of the patient (e.g., age, gender, ethnicity), as well as patient goals, concerns, expectations, requests, and/or motivations related to cosmetic treatment. In some embodiments, the patent data includes a record of previous cosmetic procedures (e.g., surgery and/or injections), including dates and any adverse effects.

Based on the patient's desired outcomes, the system determines (808) an evaluation process. In some embodiments, the evaluation process includes customized questions (e.g., 406) and/or a physical examination, the responses and results of which are saved as additional data 128 for the patient. For example, a physical examination includes an evaluation (e.g., using the evaluation model 400) of the face and neck at rest, quality of skin (e.g., whether there is sun damage, solar lentigines, redness, rhytids, thinness, and/or presence of scars), and/or impact of previous facial procedures (e.g., surgery and/or injections). In some embodiments, the examination includes an assessment of facial symmetry while the face is at rest, including one or more of forehead and facial rhytids, eyebrow height, orbital aperture height and width, cheek bone projection, lip length and vertical height, degree of nasolabial folds (NLF), MFs, and/or jowls. In some embodiments, the examination includes an assessment of platysmal band prominence (static vs. mimetic bands) of the neck.

In some embodiments, the system acquires (810) subsequent patient data based on initial results of the evaluation. For example, subsequent patient data includes additional questions 406, and/or additionally captured images 126 for assessing the face with different expressions. In some embodiments, for additionally captured images, the system prompts the patient to manipulate the upper face (e.g., scowl, raise eyebrows, smile) and/or the lower face (e.g., kiss, frown, smile) for further evaluation. For example, the system determines how animation of these facial features impacts signs of aging.

In some embodiments, the system (e.g., evaluation model 148) assesses deficient anterior malar projection, prominent tear trough, deficient submalar fullness, elongation of white upper lip, and/or volume loss in the lips.

In some embodiments, upon obtaining additional images of the patient's face (e.g., rotated to reveal oblique and/or profile contralateral angles), the system assesses flattening of the ogee curve, elongated lid-cheek junction, flattening of the cheek regions, concavity along cheeks, heaviness/sagging of cheeks, rhytids along the cheeks, loss of definition along jaw line, presence of j owls, and/or prominence of neck bands (Grade I-IV).

In some embodiments, upon obtaining additional images of the patient's face (e.g., positioned with the chin down and eyes up), the system assesses the cheek, jowls, and lid-cheek junction, hollowness along the tear trough, the effect of the head tilt on lower facial tissues, quality of transition between lower lid and cheek, degree of lower lid fat pseudoherniation, lack of structural support along midface, extent of waviness (lines and folds) along lower face, condition of oral commissures, NLFs, MFs, and/or extent of jowls.

In some embodiments, the system determines, based on the subsequently obtained patient data, that the patient is not a good candidate for injectables but is a good candidate for plastic surgery. In some embodiments, the system determines (or helps the practitioner determine) which patients should not be treated based on their answers to certain questions (e.g., because of permanent body dysmorphic disorder, or other problems).

Based on the patient data, the system determines (812) a recommended treatment plan (e.g., treatment plan 130 using treatment model 148). For example, the treatment plan specifies a particular neuromodulator to be injected throughout dictated facial regions. In some embodiments, the system determines the dictated regions of the face based on the patient's data 128 (e.g., concerns) and the recommendations of the treatment model 148. In some embodiments, the system accounts for potential anatomical obstructions, such as arteries, veins, and nerves (e.g., using anatomical model 148 as described above). In some embodiments, the system accounts for documented and learned concepts of facial beauty (e.g., using rating model 148 as described above). Example guidelines for treatment plans are described below.

In some embodiments, the system provides (814) the recommended treatment plan via output data on a user interface of a display of the system 100 or device 154. In some embodiments, the output data includes one or more computer generated facial views (e.g., frontal, 45°, and 90° views) of partial correction outcomes and/or full correction outcomes using neuromodulators and fillers (e.g., “after” images 704 using comparison model 148).

### Example Treatment Procedures and Guidance

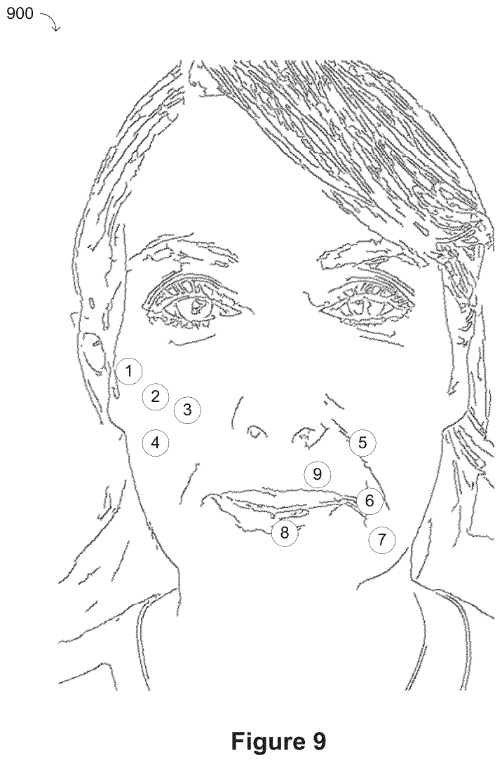

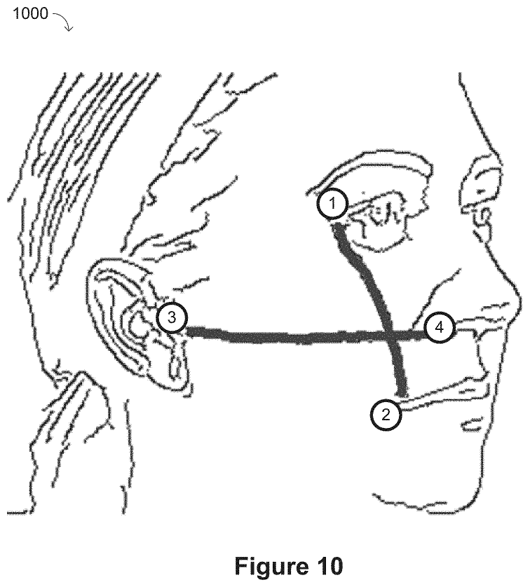

FIGS. 9-11 are diagrams of various patient images (e.g., 126) with markers provided for guidance. In some embodiments, system 100 outputs one or more of these images on a display of the system 100 or the device 154 in order to guide the practitioner during a procedure (e.g., 130).

FIG. 9 is a diagram 900 of a patient image (e.g., 126) with a plurality of markers indicating (i) injection sites, and (ii) injection sequence. The nine markers in this example indicate injection areas in a sequence from 1 (to be injected first) to 9 (to be injected last). However, other examples may specify alternative sequences depending on the particular treatment plan 130. The example sites and sequence shown in FIG. 9 include:
1: Lateral high zygomatic arch
2: Lateromedial zygomatic arch
3: Ateriomedial zygomatic arch
4: Submalar region
5: Nasolabial fold
6: Oral commissure
7: Marionette line
8: Upper/Lower lip volume and vermillion definition
9: Upper/Lower white lip rhytids

In some embodiments, the treatment plan includes guidance for the practitioner. For example:
Inject to add volume and provide lift laterally along zygomatic arch before anteromedial and submalar regions. This (i) avoids overfilling the submalar region and (ii) optimizes the ogee curve.
After treating the midface, assess lower-face wrinkles and folds. Correcting midface volume loss often impacts the approach to treatment of the lower face.

In some embodiments, the treatment plan includes guidance for marking the face with lines (e.g., Hinderer's lines), as shown in diagram 1000 of FIG. 10. The lines in this example are marked from (1) the lateral canthus to (2) the oral commissure; and from (3) the tragus to (4) the upper alar lobule, with the lid-cheek junction being marked as an upper boundary. The four points in this example are:
1: Anchor point of the zygomatic arch, lateral to the zygomatic suture
2: The most prominent point of the zygomatic arch, medial to the zygomatic suture
3: Lateral to the infraorbital foramen
4: Submalar hollow

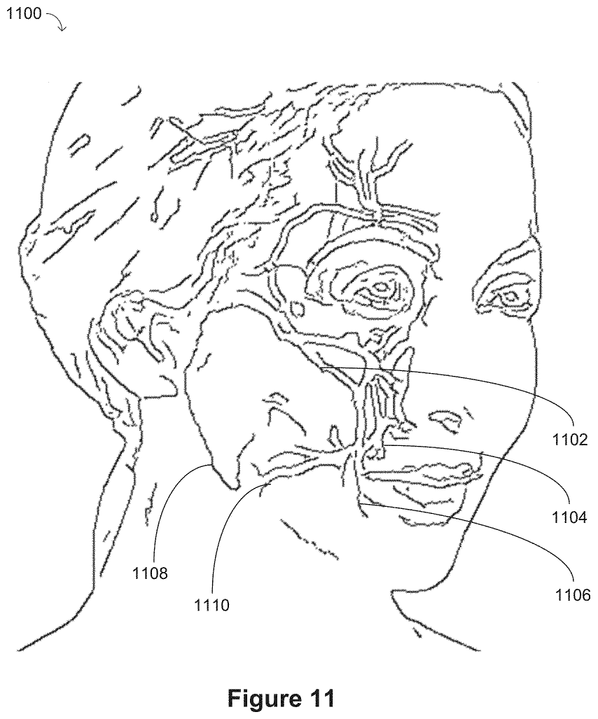

In some embodiments, the treatment plan includes guidance for avoiding anatomical obstructions, as shown in diagram 1100 of FIG. 11. In some embodiments, the system generates this type of output image based on anatomical model 148 (FIG. 5). For example, diagram 1100 corresponds to anatomy image 504. The guidance includes markers (e.g., 1102-1110) that point out areas to avoid or areas around which to use caution. Example obstructions include the transverse facial artery, facial and angular vessels, the infraorbital neurovascular bundle, the angular artery and vein, and the parotid gland and duct.

In some embodiments, the treatment plan includes treatment goals and cautionary messages for each injection site. Referring back to FIG. 9, for example, the system provides the following guidance in accordance with some embodiments.
1: Lateral high zygomatic arch
   Anatomical treatment goals: Add supraperiosteal structural support and volume to lateral part of suborbicularis fat pad to lift cheek laterally; Establish an anchor point and fuller, rounder profile in upper cheek.
   Anatomical caution: Transverse facial artery and facial nerve run along inferior margin of zygomatic arch.
   Depth: Supraperiosteal or deep subcutaneous.
2: Lateromedial zygomatic arch
   Anatomical treatment goals: Add supraperiosteal structural support and volume to lateral part of the suborbicularis fat pad to lift and fill projection point of cheekbone; Establish transition between cheekbone and frontal cheek.
   Anatomical cautions: Transverse facial artery and facial nerve run along inferior margin of zygomatic arch.
   Depth: Supraperiosteal or deep subcutaneous.
3: Ateriomedial zygomatic arch
   Anatomical treatment goals: Restore volume to medial part of suborbicularis fat pad; Gently correct deflation in the apple of the cheek.
   Anatomical cautions: Facial and angular vessels, which pass through anteromedial cheek; Infraorbital neurovascular bundle, located deep to subcutaneous plane extending from infraorbital foramen; Angular artery and vein, located medial to this area near alar lobule. This area is not to be injected.
   Depth: Subcutaneous and superficial to infraorbital foramen.
4: Submalar region
   Anatomical treatment goals: Restore volume in deep medial cheek fat pad; Correct appearance of atrophy and smooth concavity between cheekbone and lower jaw.
   Anatomical cautions: Transverse facial artery and vein, which run along the inferior margin of the zygomatic arch at the transition between zygomaticomalar and submalar regions; Parotid gland and duct, located posteriorly in this area.
   Depth: Subcutaneous.
5: Nasolabial fold
   Anatomical treatment goals: Fill nasolabial folds
   Anatomical cautions: Facial artery and vein; Buccal nerve; Superior and inferior labial arteries; Angular artery and vein, located near superior nasolabial fold next to alar lobule; This area is not to be injected.
6: Oral commissure
   Anatomical treatment goals: Smooth oral commissures
   Anatomical cautions: Facial artery and vein; Buccal nerve; Superior and inferior labial arteries; Angular artery and vein, located near superior nasolabial fold next to alar lobule; This area is not to be injected.
7: Marionette line
   Anatomical treatment goals: Reduce marionette lines
   Anatomical cautions: Facial artery and vein; Buccal nerve; Superior and inferior labial arteries; Angular artery and vein, located near superior nasolabial fold next to alar lobule; This area is not to be injected.
8: Upper/Lower lip volume and vermillion definition
   Anatomical treatment goals: Smooth vertical lip lines Anatomical cautions: Facial artery and vein; Buccal nerve; Superior and inferior labial arteries; Angular artery and vein, located near superior nasolabial fold next to alar lobule; This area is not to be injected.

### Additional Implementations

In addition or in the alternative to the embodiments described above, the following discussion includes additional implementations of the computer system 100.

In some embodiments, a reference image database 152 includes a plurality of facial images that the computing platform uses for comparison with one or more images of a patient's face (e.g., while using a treatment model 148 to determine a treatment plan 130). By using rating data 600, the computing platform generates treatment plans that increase patients' beauty. In some embodiments, database 152 is kept current by reviewing images of the faces of celebrities, models, and/or winners of various beauty contests in different parts of the world to maintain currency with what are considered the best up-to-date appearances.

In some embodiments, by obtaining both before and after photos (e.g., comparison data 700), the machine learning module learns with experience which outcomes are most completely and/or accurately obtained, by comparing an actual “after” image to the predicted “after” image 704.

In some embodiments, a reference image database 152 includes images depicting aging changes. The computing platform (e.g., a model 148) selects the best opportunities for changes based on the patient's age. In some embodiments, the computing platform (e.g., a model 148) identifies which changes will be best assuming the patient may have no further work done after the current session. Alternatively, the computing platform (e.g., a model 148) identifies which changes will be best assuming the patient's face will be enhanced by future treatments.

In some embodiments, the computing platform recommends a customized skin care program (e.g., in addition to the treatment data 130), including laser and/or important dermatological treatments for faces that would benefit. This aspect of the system utilizes the knowledge of other clinical specialists such as a dermatologist or aesthetician, bringing a multiple-specialist consultation to the computing platform utilization.

In some embodiments, the computing platform (e.g., a model 148) forecasts future facial degradations that might be averted through actions or different treatments (e.g., procedures 130).

In some embodiments, the computing platform (e.g., model 148) compares one or more images of the patient's face at the time of treatment to corresponding image(s) of the patient's face at a point in time subsequent to treatment (e.g., months or years after cosmetic injections) to determine if additional treatment is necessary. The computing platform may use multiple instances of image data 126 for a given patient (acquired over time) as machine learning inputs. By comparing the patient's face over time, the computing platform may not only determine if additional treatment is necessary based on the patient's response to past treatments, but may also determine an exact treatment plan 130 (as described above) based on the patient's response to past treatments.

### Spatial Measurements and Mathematical Standards

In some embodiments, the computing platform defines an ideal beautiful look using a mathematical definition of a beautiful face and generates treatment plans based on differences between patient facial features and corresponding mathematical standards. FIGS. 12A-14B include examples of such standards. These standards are exemplary in nature, and those skilled in the art will appreciate from the present disclosure that various other mathematical measurements have not been illustrated for the sake of brevity, and so as not to obscure more pertinent aspects of the example implementations disclosed herein. As such, examples described herein referring to specific facial features and measurements should be construed as also being applicable to other facial features and measurements.

Referring to FIG. 12A, the facial height is divided in three main thirds. The upper facial third extends from the hairline to the nasion (N) or glabella (i.e., from the top of the forehead to the bridge of the nose). The middle third extends from the nasion (N) to the subnasale (SN) or columella (i.e., from the bridge of the nose to the base of the nose or the upper lip). The lower third extends from the subnasale (SN) to the pogonion (Pog) (i.e., from the base of the nose to the deepest point of the chin prominence). The lower third is subdivided into an upper third that extends from the subnasale (Sn) to the lip commissure (lower lip), and a lower two thirds that extends from the lip commissure to the pogonion (Pog).

A horizontal line (referred to as the Frankfurt horizontal) extends through the porion (P) to the orbitale (Or), and is an important line for facial measurements. The porion (P) is the point on the human skull located at the upper margin of each ear canal and underlying the tragus (a prominence on the inner side of the external ear, in front of and partly closing the passage to the hearing organs). The orbitale (Or) is the lowest point on the lower edge of the cranial orbit.

A vertical line extends from the nasion (N) to and through the subnasale (Sn). The nasion is the bridge of the nose—the midline bony depression between the eyes where the frontal and two nasal bones meet, just below the glabella. The subnasale is a point where the nasal septum (which separates the left and right airways of the nasal cavity, dividing the two nostrils) and the upper lip meet in the midsagittal plane (the median plane that divides the body into two parts). The most anterior chin point pogonion (Pog) is located slightly posterior to that line.

While the aforementioned lines and ratios may represent mathematical proportions describing what could otherwise be subjectively referred to as “beauty,” a face may still be considered to be beautiful with certain deviations (less than respective thresholds) from those lines and ratios.

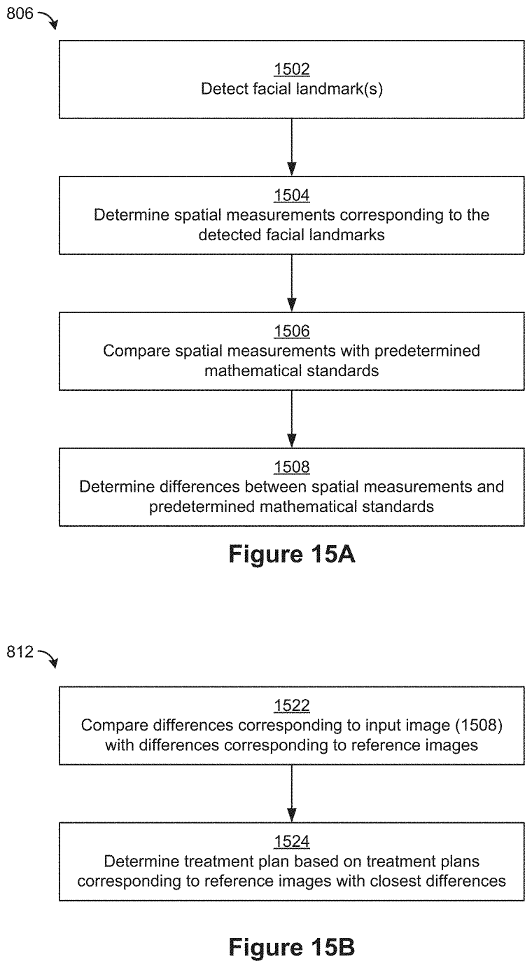

These lines and the angles between the lines may be projected according to the positions of the aforementioned facial components (P, Or, N, Sn, Pog, and so forth), with the ideal proportion of space between the horizontal lines being ⅓ or ⅔ as described above, and the ideal of angles between vertical and horizontal lines being right angles. Differences between the ideal spacing/angles and actual spacing/angles may be used as bases for determining treatment plans (as described in more detail below with reference to FIGS. 15A-15B).

Referring to FIG. 12B, the width of the face is divided into a plurality of segment. Each segment is a mathematical proportion of facial measurements. For example, the segment representing the width of the base of the nose may be compared to the two eye width segments, with an ideal nose to eye proportion being 1:3 (i.e., the width of the base of the nose is equal to three eye widths). The nose segments may be defined based on lines extending tangential to the base of the nose vertically oriented straight up and down. The vertical nose lines connect with the medial campus (corner) of each eye, while the vertical eye lines include a set of the aforementioned lines (one for each eye) and a set of lines extending tangential from the lateral corner of each eye.

These vertical lines may be projected according to the positions of the nose and eye components (i.e., the ends of the base of the nose, the medial campus of each eye, and the lateral corner of each eye) and according to the ends of the face corresponding to the positions of the ears, with the ideal proportion of space between each line being ⅕ (i.e., equally spaced). Differences between the ideal spacing and actual spacing may be used as bases for determining treatment plans (as described in more detail below with reference to FIGS. 15A-15B).

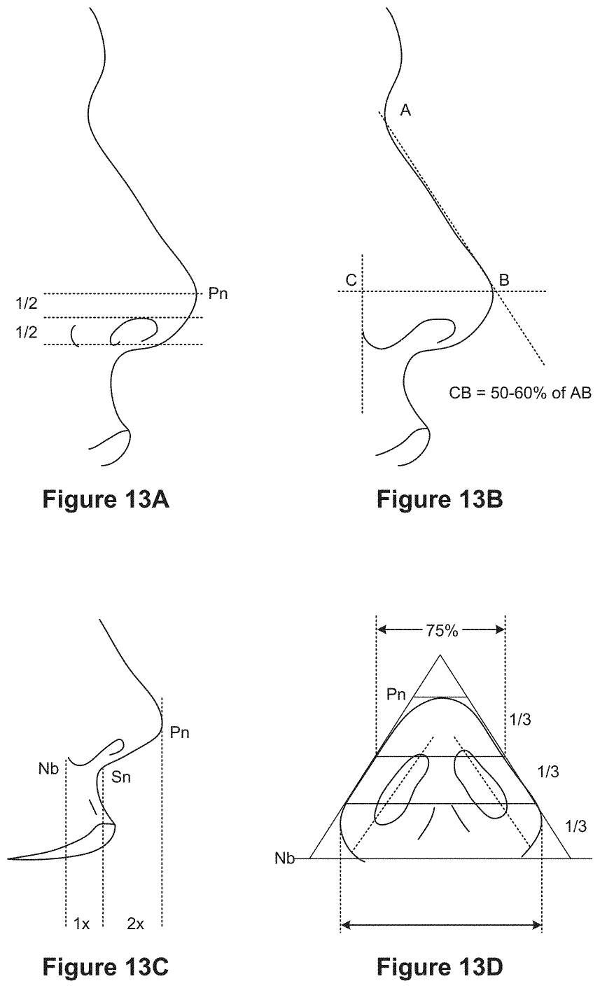

Referring to FIG. 13A, the area around the nostril is divided in two segments by projecting a horizontal intersecting the pronasale (Pn) or nose tip, a horizontal line tangential to the top of the nostril, and a horizontal line tangential to the bottom of the nostril, in order to evaluate nostril show. Ideal proportions may be defined as being equivalent, such that the distance between the pronasale line and the nostril bottom line is equally split in half with the top nostril line. Differences between the ideal spacing and actual spacing may be used as bases for determining treatment plans (as described in more detail below with reference to FIGS. 15A-15B).

Referring to FIG. 13B, a nasal projection line (CB) and nasal length line (AB) are projected about the nose. The ideal CB to AB ratio may be defined as anywhere between 0.5 and 0.6 (i.e., CB is 50%-60% of the length of AB). Differences between the ideal ratio and the actual ratio may be used as bases for determining treatment plans (as described in more detail below with reference to FIGS. 15A-15B).

Referring to FIG. 13C, a horizontal assessment of nasal projection may be considered by dividing the nose into two segments, defined by a first vertical line tangential to the pronasale (Pn), a second vertical line intersecting the subnasale (Sn), and a third vertical line intersecting the nasal bone (Nb). The ideal Pn-Sn to Sn-Nb ratio is 2:1 (i.e., the Pn-Sn segment is twice as long as the Sn-Nb segment). Differences between the ideal ratio and the actual ratio may be used as bases for determining treatment plans (as described in more detail below with reference to FIGS. 15A-15B).

Referring to FIG. 13D, a nasal base (sometimes referred to as “worm's eye”) assessment of the nose may be considered by projecting three straight lines respectively tangential to the nasal bone (Nb) and each side of the nose, forming an alar base. The alar base is ideally shaped like an isosceles triangle (a triangle having two sides of equal length). Differences between the ideal shape and proportions of the alar base and the actual shape and proportions of the alar base may be used as bases for determining treatment plans (as described in more detail below with reference to FIGS. 15A-15B).

In addition, with reference to FIG. 13D, the base view of the nose may be divided into three segments, defined by a first line about the nasal bone (Nb), a second line bounded at the widest edge of the sides of the nose, a third line tangential to the ends of the nostrils closest to the nose tip, and a fourth line at the pronasale (Pn). The ideal ratio between the segment closest to the nose tip (between the nostril ends and the pronasale) and the two other segments is 1:2 (i.e., the nose tip segment is one-third the width of the other two segments combined). Differences between the ideal ratio and the actual ratio may be used as bases for determining treatment plans (as described in more detail below with reference to FIGS. 15A-15B).

In addition, with reference to FIG. 13D, the line projected about the nostril tips is ideally 75% of the length of the line projected between the widest edges of the sides of the nose. Differences between the ideal proportions and the actual proportions may be used as bases for determining treatment plans (as described in more detail below with reference to FIGS. 15A-15B).

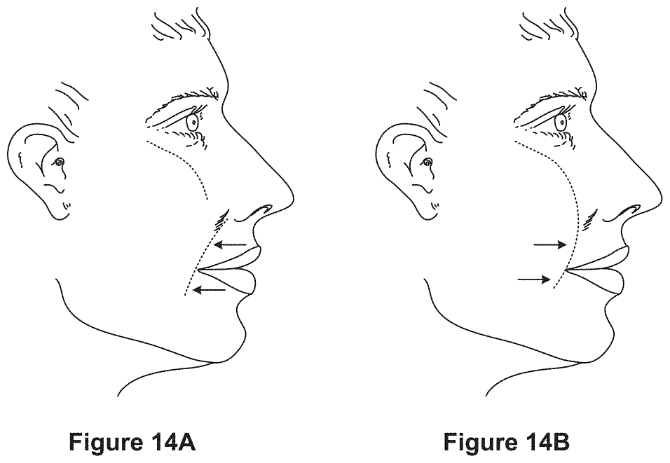

Referring to FIGS. 14A-14B, the contour comprising the cheekbone, nasal base, and lip curve may be assessed. The curve is ideally uninterrupted and smooth in an individual with ideal facial proportions. In FIG. 14A, the interruption of the curve (as depicted by the arrows) indicates maxillary and mandibulae anteroposterior deficiency. Following maxillary and mandibular advancement, as shown in FIG. 14B, the curve is uninterrupted and smooth (as depicted by the arrows). Differences between the ideal curve and the actual curve may be used as bases for determining treatment plans (as described in more detail below with reference to FIGS. 15A-15B).

The examples of spatial measurements and mathematical standards described above with reference to FIGS. 12A-14B are exemplary in nature, and those skilled in the art will appreciate from the present disclosure that various other mathematical measurements may be used as bases for spatial measurements and mathematical standards.

In some implementations, a nonlimiting list of such measurements include those that may be obtained via a profile evaluation of the patient, including measurements detailing the antero-posterior position of the maxilla, the antero-posterior position of the mandible, nasal size, contours of the cheeks, lip support, lip competence, the size of the mandibular angle, measurements of facial soft tissues (e.g., amount, tension, and so forth), and orthognathic measurements.

In some implementations, a nonlimiting list of such measurements include those that may be obtained via a frontal view (en face) evaluation of the patient, including facial midline, symmetry, muscle activity of the lower lip and chin, tooth to lip relationship, lip length, facial contour, head to body proportion, and orthognathic measurements.

In some implementations, the measurements described above may be obtained by the use of a three dimensional (3D) camera. The camera may project a grid onto the face and take a plurality of images using frontal, oblique, and side views (or just frontal and side views). Each small region of the face in each image may be dissected and analyzed according to the mathematical measurements described above. Specifically, the measurements of the patient's face (referred to as actual measurements) may be compared to the measurements corresponding to a mathematically ideal face (referred to as ideal measurements), as described above. In this comparison, facial landmarks (aesthetic facial units) may be used to compare the actual measurements to the ideal measurements, as described above with reference to FIGS. 12A-14B (e.g., Pog, Or, N, Sn, Pn, and so forth). In some implementations, detection of the facial landmarks is a computer vision task in which a model predicts key points representing regions or landmarks on the patient's face. These landmarks are inputted into the computer system, and differences between the actual measurements and ideal measurements may be evaluated aesthetically.

In some implementations, the evaluation may initially start with experts and their opinions on faces, and eventually use machine learning (as described above) to relate those to the face at hand. A set of recommendations may be proposed based on outputs of the machine learning evaluation. The machine learning models may be trained using the mathematical differences as inputs and expert recommendations as input labels, and the outputs of the machine learning models may be recommendations based on the mathematical differences.

In an alternative approach, the mathematical description of the face may be compared to the ideal face without the use of machine learning. Such implementations would not have to deal with conflicting expert opinions, as they would be grounded in impartial mathematical principles.

As a result of the comparisons, a treatment plan may be proposed (as described above) specifying injection characteristics (e.g., type, sequence, and/or location) for clinician injectors to utilize. The exact locations for the injections may be presented, optionally overlaying a grid. While a final outcome may be determined, the goals for initial treatments may be incremental, taking the patient only part of the way to the ideal they can reach (e.g., with plastics and/or fillers, but not plastic surgery). In some implementations, the aforementioned mathematical differences may be translated to an action plan including suggestions for how to achieve more aesthetic proportions (e.g., bring out the jaw if the maxilla is too far forward, or push it back if the maxilla is too far back).

FIGS. 15A-15B are flow diagrams illustrating operations of method steps 806 and 812, respectively, as described above with reference to method 800 (FIG. 8). Alternatively, operations 806 and 812 in FIGS. 15A-15B may be performed without some or all of the other operations in method 800 (FIG. 8). In either case, the operations in FIGS. 15A-15B may be performed at a computing platform 100 (also referred to herein as a computer system), a local or remote image database 152, and/or a practitioner device 154. For example, instructions for performing the method are stored in the memory 102 and executed by the processor(s) 104 of the computer system 100. In some embodiments, part or all of the instructions for performing the operations in FIGS. 15A-15B are stored in memory and executed by processor(s) of the practitioner device 154.

In operation 1502, the computer system detects facial landmarks in images captured with, for example, a 3D camera. Examples of facial landmarks are described above with reference to at least FIGS. 12A-14B (e.g., Pog, Or, N, Sn, Pn, and so forth). In some implementations, detection of the facial landmarks is a computer vision task in which a model predicts key points representing regions or landmarks on the patient's face. These landmarks are inputted into the computer system for further processing.

In operation 1504, the computer system determines spatial measurements corresponding to the detected facial landmarks. Examples of spatial measurements are described above with reference to at least FIGS. 12A-14B (e.g., distance between facial landmarks, proportions and ratios involving facial landmarks and corresponding distances between them, shapes of facial features and landmarks, and so forth).

In operation 1506, the computer system compares the spatial measurements (actual measurements) with predetermined mathematical standards (ideal measurements) corresponding to mathematically ideal faces. Examples of such comparisons are described above with reference to FIGS. 12A-14B (e.g., difference between actual and ideal distances between facial landmarks, differences between actual and ideal proportions and ratios involving facial landmarks and corresponding distances between them, differences in actual and ideal shapes of facial features and landmarks, and so forth).

In operation 1508, the computer system determines mathematical differences between the spatial measurements (actual measurements) and the predetermined mathematical standards (ideal measurements). For example, based on the comparison of the measured ratio of two segments to the ideal ratio of the two segments, a difference between the two ratios (measured and ideal) is determined. For example, with reference to FIG. 13B, the CB segment may be 75% of the AB segment as measured on the patient's face. Compared to an ideal percentage of 55%, the difference between measured and ideal percentages is 20%.

In operation 1522, the computer system compares the differences corresponding to the input images of the patient (the differences determined in operation 1508 based on actual measurements of the patient's face) with differences corresponding to reference images (differences measured on faces of people other than the patient). For example, a difference of 20% between measured and ideal proportions of the CB and AB segments for the patient may be compared to reference images of other patients having a 20% difference between their measured and ideal proportions of the CB and AB segments. While the ideal proportions are the same across all images (the input images and the reference images), the measured proportions are based on the actual facial features of the patients in each image (the patient in the input images and the patients in the reference images).

In operation 1524, the computer system determines a treatment plan for the patient based on treatment plans corresponding to the reference images with the closest differences. For example, reference images having a 20% difference between their measured and ideal proportions of the CB and AB segments correspond to treatment plans that were used on the respective patients associated with those images. Thus, a treatment plan for the current patient may be determined based on the treatment plans corresponding to those reference images (i.e., corresponding to the respective patients associated with those reference images).

Thus, in an illustrative example of the concepts described above with reference to the operations described in FIGS. 8 and 15A-15B, a computer system obtains an input image of a face of a user (also referred to as the patient); compares, using a pattern recognition process, image data (e.g., facial landmarks of the user and characteristics thereof, including shapes, distances, ratios, proportions, and so forth) of the input image to corresponding image data of a plurality of reference images (e.g., facial landmarks of individuals other than the user and characteristics thereof). Each of the plurality of reference images includes a face of an individual other than the user.

The computer system determines, based on the input image and the comparing of the image data (facial landmarks and characteristics thereof) of the input image to the corresponding image data (facial landmarks and characteristics thereof) of the plurality of reference images, a treatment plan. The treatment plan includes injecting agent characteristics, including type, amount, injecting locations, and/or injecting sequence. The computer system displays the treatment plan on a user interface of the electronic computer system.

The computer system detects a plurality of facial landmarks on the input image of the face (as described in operation 1502), determines one or more spatial measurements corresponding to the plurality of facial landmarks (as described in operation 1504), compares the one or more spatial measurements to corresponding predetermined mathematical standards representing ideal facial characteristics (as described in operation 1506), and based on the comparing, determines one or more differences between the spatial measurements and the corresponding predetermined mathematical standards (as described in operation 1508). The image data of the input image (associated with the user) includes the one or more differences between the spatial measurements and the corresponding predetermined mathematical standards; and the corresponding image data of the plurality of reference images (associated with individuals other than the user) includes respective differences between spatial measurements corresponding to respective reference images of the plurality of reference images and the corresponding predetermined mathematical standards.

In some implementations, the pattern recognition process uses a model refined by unsupervised or adversarial training. Inputs of the model include the plurality of reference images (associated with individuals other than the user) and respective differences between spatial measurements corresponding to respective reference images of the plurality of reference images (actual differences) and the corresponding predetermined mathematical standards (ideal differences). The input labels of the model include treatment plans (e.g., comprising injecting agent amounts and/or injection locations) corresponding to respective reference images of the plurality of reference images.

In some implementations, the one or more spatial measurements include one or more measurements described above with reference to FIGS. 12A-14B and the following discussion (e.g., including one or more orthognathic measurements), and the corresponding predetermined mathematical standards include corresponding ideal measurements (e.g., including predetermined orthognathic standards).

In some implementations, with reference to FIG. 12A, the plurality of facial landmarks on the input image of the face include two or more landmarks selected from the group consisting of a porion, an orbitale, a nasion, a subnasale, and a pogonion; the one or more spatial measurements include a distance, angle, or proportion involving (i) a first line bisecting the porion and the orbitale, and (ii) a second line bisecting the nasion, the orbitale, the subnasale, or the pogonion; and the predetermined mathematical standards include a predetermined distance, angle, or proportion involving the first line and the second line.

In some implementations, with reference to FIG. 12A, the plurality of facial landmarks on the input image of the face include a hairline, a glabella, a columella, and a pogonion; the one or more spatial measurements include a first distance between the hairline and the glabella, a second distance between the glabella and the columella, and a third distance between the columella and the pogonion; and the predetermined mathematical standards include a predetermined proportion involving the first distance, the second distance, and the third distance.

In some implementations, with reference to FIG. 12B, the plurality of facial landmarks on the input image of the face include a left eye, a right eye, a nose, and a mouth; the one or more spatial measurements include a first distance between a left edge of the face and the left eye, a second distance between two ends of the left eye, a third distance between the left eye and the right eye or between two ends of the nose, a fourth distance between two ends of the right eye, and a fifth distance between the right eye and a right edge of the face; and the predetermined mathematical standards include a predetermined proportion involving the first distance, the second distance, the third distance, the fourth distance, and the fifth distance.

In some implementations, with reference to FIG. 13A, the plurality of facial landmarks on the input image of the face include a nostril bottom, a nostril top, and a nose tip; the one or more spatial measurements include a first distance between the nostril bottom and the nostril tip and a second distance between the nostril top and a line bisecting the nose tip; and the predetermined mathematical standards include a predetermined proportion involving the first distance and the second distance.

In some implementations, with reference to FIG. 13B, the plurality of facial landmarks on the input image of the face include a nasal projection and a nasal length; the one or more spatial measurements include a ratio of the nasal projection to the nasal length; and the predetermined mathematical standards include a predetermined ratio of the nasal projection to the nasal length.

In some implementations, with reference to FIG. 13C, the plurality of facial landmarks on the input image of the face include a nasal base, a subnasale, and a nose tip; the one or more spatial measurements include a first distance between the nasal base and the subnasale and a second distance between the subnasale and a line bisecting the nose tip; and the predetermined mathematical standards include a predetermined proportion involving the first distance and the second distance.

In some implementations, with reference to FIG. 13D, the plurality of facial landmarks on the input image of the face include a nasal base; the one or more spatial measurements include a shape of the nasal base; and the predetermined mathematical standards include a predetermined shape of the nasal base.

In some implementations, with reference to FIG. 14, the plurality of facial landmarks on the input image of the face include a cheekbone, a nasal base, and a lip; the one or more spatial measurements include a curve contour of the cheekbone, the nasal base, and the lip; and the predetermined mathematical standards include a predetermined curve contour of the cheekbone, the nasal base, and the lip.

Thus, the computing platform in the implementations described above utilize visual sensors to gather facial data in order to develop facial recognition and further utilize machine learning to understand concepts of facial youthfulness and facial beauty. The platform combines that data with topographical facial analysis and the expertise of a large group of plastic surgeons, dermatologists, and other cosmetic specialists to create and recommend safe treatment protocols and algorithms for enhancing the facial features according to documented, artistic, and machine-learned concepts of youth and facial beauty grounded in mathematical principles.

### Anatomical Target Classification

In some embodiments, the computing platform analyzes images of anatomical targets, such as skin lesions, and compares them over time to determine if the targets have changed. The computing platform further compares the images of lesions (associated with a given patient) to images of lesions (associated with individuals other than the given patient) in a reference library to determine if the lesions are of concern (e.g., skin cancer). Such embodiments may be used for patients who have routine visits with a dermatologist, as well as new patients who have lesions that look precarious and are invited to return for follow-up visits.

For a given patient, the electronic computer system obtains multiple images of a lesion over time and compares them. The images may capture the surface of the patient's skin (e.g., using a superficial camera). Additionally or alternatively, the images may capture underneath the surface of the patient's skin (e.g., using a dermascope). In general, the images may cover portions or all of the epidermis, dermis, and/or hypodermis in a portion of the skin including the lesion. The computer system may analyze differences between the images at the pixel level, thereby providing results to an accuracy that may not be possible using hand-held measuring tools.

The computer system may detect changes in size, depth, and/or color of the lesion, or any other factor indicating the potential for alteration of the lesion over time. In some implementations, a grid may be projected onto the region of skin in which the lesion is located, and the computer system may use the grid to enhance the accuracy of its measurements.

In some implementations, image database 152 of the computing platform stores a plurality of series of images of lesions captured over time (referred to herein as reference images). Each individual series of the plurality of series includes at least a first image of a lesion captured at a first time and a second image of the lesion captured at a second time subsequent to the first time. The first and second images are captured at least one month apart from each other, and preferably at least three months apart in order to provide enough time for temporal alterations in the lesion to be discernable across the series of images. Each series of reference images in the database is associated with a label.

In some implementations, the labels are classifications of the respective lesions in each respective series of reference images. Example classifications include “cancer” and “not cancer.” In some implementations, more specific “cancer” classifications may include cancer types such as “basal cell carcinoma,” “squamous cell carcinoma,” “Merkel cell cancer,” “melanoma,” and so forth. In some implementations, labels may include other details describing the type of lesion, such as “blister,” “macule,” “nodule,” “papule,” “rash,” “wheal,” “crust,” “scale,” “scar,” “skin atrophy,” “ulcer,” and so forth. These labels are initially assigned by plastic surgeons, facial plastic surgeons, oculoplastic surgeons, dermatologists, laser specialists, anatomists, and/or research and development experts in the fields of skin disease.

In some implementations, the labels are growth determinations of the respective lesions in each respective series of reference images. Example growth determinations include “growth” and “no growth.” Since images of the same lesion captured over time may not always be taken from the same distance and angles, the lesion may be a different size in each image. Thus, it is important to determine if the difference in size is due to growth of the lesion, or due to difference in other factors such as camera capture distance or angles. By registering the lesion to other landmarks on the skin, the computer system may determine whether the size difference is a result of growth of the lesion or the result of camera capture factors. Example landmarks include anatomical feature such as hair follicles or wrinkles, or digital features such as projected gridlines. These labels may be initially assigned by plastic surgeons, facial plastic surgeons, oculoplastic surgeons, dermatologists, laser specialists, anatomists, and/or research and development experts in the fields of skin disease.

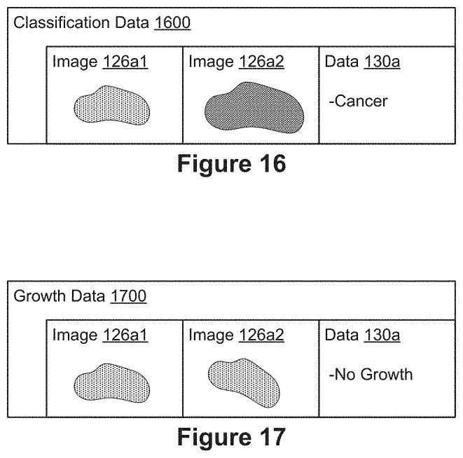

FIGS. 16-17 are diagrams showing data structures for machine learning processes in accordance with some embodiments. Embodiments of the machine learning module 140 train one or more models 148 in accordance with each figure, as explained in more detail below. In some embodiments, the input images that make up the respective data structures are stored in a local or remote database 152. Alternatively, the input images that make up the respective data structures are stored in memory 102 of the computing platform 100. In any case, the computing platform inputs the data in a respective structure into a training module (e.g., 142, 144, or 146) for development of a respective model 148, as described below.

FIG. 16 is a diagram of a classification data structure 1600 corresponding to patient 124a in FIG. 1 in accordance with some embodiments. For each patient, a series of images including at least a first image 126a1 and a second image 126a2 are stored. Each image in the series of images includes an anatomical target, such as a lesion, on a portion of skin of a patient. While lesions are used as the anatomical target in the present discussion, the concepts described herein also apply to any other anatomical feature that may be visible (or seemingly invisible) on the skin of a patient and can potentially be implicated in an adverse health outcome (e.g., cancer). Each series of images includes an anatomical target on a different patient. When training a model 148 for determining whether a lesion is cancerous, or a degree of likelihood of cancer, or a type of cancer, or a type of lesion (as discussed above), classification data 130a is also stored and associated with the series of images 126a1-126a2 as a label for those images. In some embodiments, images 126a1-126a2 includes a plurality of images of the patient, including images taken from different angles, and/or images showing different areas of the portion of skin including the anatomical target. For training purposes, the input (reference) images 126 serve as machine learning input, and the corresponding classification data 130 serves as an input label for that machine learning input. With a set of inputs (e.g., hundreds, thousands, or more), machine learning module 140 generates a classification model 148 for classifying anatomical targets in new series of images 126.

FIG. 17 is a diagram of a growth data structure 1700 corresponding to patient 124a in FIG. 1 in accordance with some embodiments. For each patient, a series of images including at least a first image 126a1 and a second image 126a2 are stored. Each image in the series of images includes an anatomical target, such as a lesion, on a portion of skin of a patient. Each series of images includes an anatomical target on a different patient. When training a model 148 for determining whether a lesion has grown over time (as discussed above), growth data 130a is also stored and associated with the series of images 126a1-126a2 as a label for those images. In some embodiments, images 126a1-126a2 includes a plurality of images of the patient, including images taken from different angles, and/or images showing different areas of the portion of skin including the anatomical target. For training purposes, the input (reference) images 126 serve as machine learning input, and the corresponding growth data 130 serves as an input label for that machine learning input. With a set of inputs (e.g., hundreds, thousands, or more), machine learning module 140 generates a growth model 148 for characterizing the growth (or lack thereof) of anatomical targets in new series of images 126.

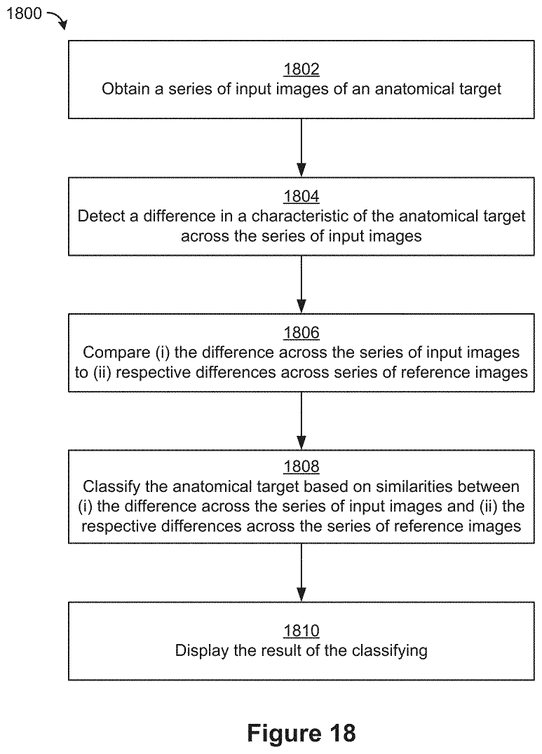

FIG. 18 is a flow diagram illustrating a method 1800 for classifying an anatomical target in accordance with some embodiments. The method is performed at a computing platform 100 (also referred to herein as a computer system), a local or remote image database 152, and/or a practitioner device 154. For example, instructions for performing the method are stored in the memory 102 and executed by the processor(s) 104 of the computer system 100. In some embodiments, part or all of the instructions for performing the method are stored in memory and executed by processor(s) of the practitioner device 154.

The system obtains (1802) a series of input images of an anatomical target (e.g., a lesion) on a portion of skin of a user, wherein the series of input images includes at least two input images (e.g., 126a1 and 126a2) captured at least one month apart from each other.

The system detects (1804) a difference in a characteristic of the anatomical target across the series of input images. In some implementations, the characteristic of the anatomical target is a spatial measurement (e.g., one or more of size, depth, length, width, diameter, circumference, depth, and/or other quantitative feature) or a spectral measurement of the anatomical target (e.g., one or more of color, texture, pattern, and/or other visual feature). In some implementations, the difference in the characteristic of the anatomical target is a difference in any of the aforementioned spatial or spectral measurements (e.g., size, depth, color, etc.) of the anatomical target over time.

The system compares (1806), using a pattern recognition process, (i) the difference in the characteristic of the anatomical target across the series of input images (i.e., images of the patient over time) to (ii) respective differences in characteristics of anatomical targets across respective series of reference images (i.e., images of individuals other than the patient over time), wherein each of the respective series of reference images includes a portion of skin of an individual other than the user. For example, differences between reference images 126a1 and 126a2 include size (the lesion in image 126a2 is larger than the lesion in image 126a1) and color (the lesion in image 126a2 is darker than the lesion in image 126a1).

The system classifies (1808) the anatomical target on the portion of skin of the user based on similarities between (i) the difference in the characteristic of the anatomical target across the series of input images and (ii) at least one difference of the respective differences in characteristics of anatomical targets across the respective series of reference images.

In some implementations, the pattern recognition process uses a model refined by unsupervised or adversarial training (as described above with reference to the data structures in FIGS. 16-17). In some implementations, inputs of the model include a plurality of series of reference images (e.g., 126a1-126a2), including the respective series of reference images, and input labels of the model include classifications of anatomical targets included in each reference image of the plurality of series of reference images (e.g., data 130). In some implementations, the classifications include at least one cancer-related classification (as described above with reference to FIG. 16) or at least one growth-related classification (as described above with reference to FIG. 17).

In some implementations, the anatomical target on the portion of skin of the user is a lesion, and classifying the anatomical target includes classifying the lesion as cancerous or benign, or assigning a likelihood that the lesion is cancerous (as described above with reference to FIG. 16). In some implementations, classifying the anatomical target includes classifying the lesion as having grown in size over time or having not grown in size over time (as described above with reference to FIG. 17).

The system displays (1810) (or causes to be displayed) a result of the classifying on a user interface of the electronic computer system (or on a user interface of a system communicatively coupled to the electronic computer system). The result may be the classification data 130 (e.g., “cancer,” “not cancer,” and so forth) or the growth data 130 (e.g., “growth,” “no growth,” and so froth) as discussed above with reference to FIGS. 16-17.

Thus, the computing platform in the implementations described above utilize visual sensors to gather data in order to develop anatomical target recognition and further utilize machine learning to detect and analyze changes in such targets over time and classify the targets based on the detected changes, by comparing the image data (including the changes over time) gathered for a given patient with corresponding image data (including changes of similar targets over time) for individuals other than the given patient.

### Notes Regarding the Disclosure

Reference have been made in detail to various implementations, examples of which are illustrated in the accompanying drawings. In the above detailed description, numerous specific details are set forth in order to provide a thorough understanding of the invention and the described implementations. However, the invention may be practiced without these specific details. In other instances, well-known methods, procedures, components, and circuits have not been described in detail so as not to unnecessarily obscure aspects of the implementations.

It will be understood that, although the terms “first,” “second,” etc. may be used herein to describe various elements, these elements should not be limited by these terms. These terms are only used to distinguish one element from another. For example, a first device could be termed a second device, and, similarly, a second device could be termed a first device, without changing the meaning of the description, so long as all occurrences of the first device are renamed consistently and all occurrences of the second device are renamed consistently. The first device and the second device are both devices, but they are not the same device.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

What is claimed is:

1. An electronic computer system, comprising:
one or more processors; and
memory storing one or more programs for execution by the one or more processors, the one or more programs comprising instructions for:
obtaining a series of input images of an anatomical target on a portion of skin of a user, wherein the series of input images includes at least two input images captured at least one month apart from each other;
detecting a difference in a characteristic of the anatomical target across the series of input images;
comparing, using a pattern recognition process, (i) the difference in the characteristic of the anatomical target across the series of input images to (ii) respective differences in characteristics of anatomical targets across respective series of reference images, wherein each of the respective series of reference images includes a portion of skin of an individual other than the user;
classifying the anatomical target on the portion of skin of the user based on similarities between (i) the difference in the characteristic of the anatomical target across the series of input images and (ii) at least one difference of the respective differences in characteristics of anatomical targets across the respective series of reference images; and
displaying a result of the classifying on a user interface of the electronic computer system.

2. The electronic computer system of claim 1, wherein:
the pattern recognition process uses a model refined by unsupervised or adversarial training;
inputs of the model include a plurality of series of reference images, including the respective series of reference images; and
input labels of the model include classifications of anatomical targets included in each reference image of the plurality of series of reference images.

3. The electronic computer system of claim 2, wherein the classifications include at least one cancer-related classification.

4. The electronic computer system of claim 2, wherein the classifications include at least one growth-related classification.

5. The electronic computer system of claim 1, wherein:
the anatomical target on the portion of skin of the user is a lesion; and
the instructions for classifying the anatomical target include instructions for classifying the lesion as cancerous or benign, or assigning a likelihood that the lesion is cancerous.

6. The electronic computer system of claim 1, wherein:
the anatomical target on the portion of skin of the user is a lesion; and
the instructions for classifying the anatomical target include instructions for classifying the lesion as having grown in size over time or having not grown in size over time.

7. The electronic computer system of claim 1, wherein:
the characteristic of the anatomical target is a spatial measurement or a spectral measurement of the anatomical target; and
the difference in the characteristic of the anatomical target is a difference in size, depth, or color of the anatomical target over time.

8. A method, comprising:
at an electronic computer system including one or more processors and memory storing one or more programs for execution by the one or more processors:
obtaining a series of input images of an anatomical target on a portion of skin of a user, wherein the series of input images includes at least two input images captured at least one month apart from each other;
detecting a difference in a characteristic of the anatomical target across the series of input images;
comparing, using a pattern recognition process, (i) the difference in the characteristic of the anatomical target across the series of input images to (ii) respective differences in characteristics of anatomical targets across respective series of reference images, wherein each of the respective series of reference images includes a portion of skin of an individual other than the user;
classifying the anatomical target on the portion of skin of the user based on similarities between (i) the difference in the characteristic of the anatomical target across the series of input images and (ii) at least one difference of the respective differences in characteristics of anatomical targets across the respective series of reference images; and
displaying a result of the classifying on a user interface of the electronic computer system.

9. The method of claim 8, wherein:
the pattern recognition process uses a model refined by unsupervised or adversarial training;
inputs of the model include a plurality of series of reference images, including the respective series of reference images; and
input labels of the model include classifications of anatomical targets included in each reference image of the plurality of series of reference images.

10. The method of claim 9, wherein the classifications include at least one cancer-related classification.

11. The method of claim 9, wherein the classifications include at least one growth-related classification.

12. The method of claim 8, wherein:
the anatomical target on the portion of skin of the user is a lesion; and
classifying the anatomical target includes classifying the lesion as cancerous or benign, or assigning a likelihood that the lesion is cancerous.

13. The method of claim 8, wherein:
the anatomical target on the portion of skin of the user is a lesion; and
classifying the anatomical target includes classifying the lesion as having grown in size over time or having not grown in size over time.

14. The method of claim 8, wherein:
the characteristic of the anatomical target is a spatial measurement or a spectral measurement of the anatomical target; and
the difference in the characteristic of the anatomical target is a difference in size, depth, or color of the anatomical target over time.

15. A non-transitory computer readable storage medium storing one or more programs configured for execution by an electronic computer system, the one or more programs including instructions for:
obtaining a series of input images of an anatomical target on a portion of skin of a user, wherein the series of input images includes at least two input images captured at least one month apart from each other;
detecting a difference in a characteristic of the anatomical target across the series of input images;
comparing, using a pattern recognition process, (i) the difference in the characteristic of the anatomical target across the series of input images to (ii) respective differences in characteristics of anatomical targets across respective series of reference images, wherein each of the respective series of reference images includes a portion of skin of an individual other than the user;
classifying the anatomical target on the portion of skin of the user based on similarities between (i) the difference in the characteristic of the anatomical target across the series of input images and (ii) at least one difference of the respective differences in characteristics of anatomical targets across the respective series of reference images; and
displaying a result of the classifying on a user interface of the electronic computer system.

16. The non-transitory computer readable storage medium of claim 15, wherein:
the pattern recognition process uses a model refined by unsupervised or adversarial training;
inputs of the model include a plurality of series of reference images, including the respective series of reference images; and
input labels of the model include classifications of anatomical targets included in each reference image of the plurality of series of reference images.

17. The non-transitory computer readable storage medium of claim 16, wherein the classifications include at least one cancer-related classification, or at least one growth-related classification.

18. The non-transitory computer readable storage medium of claim 15, wherein:
the anatomical target on the portion of skin of the user is a lesion; and
the instructions for classifying the anatomical target include instructions for classifying the lesion as cancerous or benign, or assigning a likelihood that the lesion is cancerous.

19. The non-transitory computer readable storage medium of claim 15, wherein:
the anatomical target on the portion of skin of the user is a lesion; and
the instructions for classifying the anatomical target include instructions for classifying the lesion as having grown in size over time or having not grown in size over time.

20. The non-transitory computer readable storage medium of claim 15, wherein:
the characteristic of the anatomical target is a spatial measurement or a spectral measurement of the anatomical target; and
the difference in the characteristic of the anatomical target is a difference in size, depth, or color of the anatomical target over time.
